(19) United States
(12) Patent Application Publication
(10) Pub. No.: US 2011/0112200 A1
(43) Pub. Date: May 12, 2011

(54) SOLID STATES OF O-DESMETHYLVENLAFAXINE SALTS

(76) Inventors: Valerie Niddam-Hildesheim, Kadima (IL); Tamar Nidam, Yehud (IL); Eli Lancry, Ashdod (IL)

(21) Appl. No.: 12/991,069

(22) PCT Filed: Jun. 16, 2009
(86) PCT No.: PCT/US2009/047501

§ 371 Date: Dec. 20, 2010

Related U.S. Application Data
(60) Provisional application No. 61/061,864, filed on Jun. 16, 2008.
(60) Provisional application No. 61/081,314, filed on Jul. 16, 2008.
(60) Provisional application No. 61/089,696, filed on Aug. 18, 2008.

(51) Int. Cl. A61K 31/137; C07C 215/64; C07C 309/04; A61P 25/24

(52) U.S. Cl. 514/649; 562/597; 564/336; 562/584; 562/595; 562/114; 562/470; 562/585; 562/508; 562/582; 562/590

(57) ABSTRACT

The present invention provides crystalline and amorphous salts of O-desmethylvenlafaxine and processes for preparing said salts of O-desmethylvenlafaxine. The present invention further provides pharmaceutical compositions comprising said salts of O-desmethylvenlafaxine.

### CROSS REFERENCE TO RELATED APPLICATIONS

The present invention claims the benefit of the following U.S. Provisional Patent Application Nos. 61/061,864, filed Jun. 16, 2008; 61/081,314, filed Jul. 16, 2008; and 61/089,696, filed Aug. 18, 2008. The contents of these applications are incorporated herein by reference.

### FIELD OF INVENTION

The present invention is directed to solid states O-desmethylvenlafaxine in several salt forms and methods of preparation thereof

### BACKGROUND OF THE INVENTION

Venlafaxine, (±)-1-[2-(Dimethylamino)-1-(4-ethyoxyphenyl)ethyl]cyclo-hexanol, having the following formula

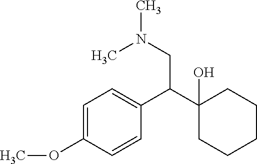

is the first of a class of anti-depressants. Venlafaxine acts by inhibiting re-uptake of norepinephrine and serotonin, and is an alternative to the tricyclic anti-depressants and selective re-uptake inhibitors.

O-desmethylvenlafaxine (ODV), chemically named 4-[2-(dimethylamino)-1-(1-hydroxycyclohexyl)ethyl]phenol having the following formula,

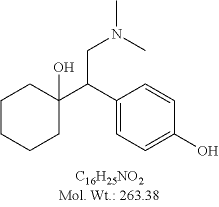

is a major metabolite of venlafaxine and has been shown to inhibit norepinephrine and serotonin uptake. Klamerus, K. J. et al., “Introduction of the Composite Parameter to the Pharmacokinetics of Venlafaxine and its Active O-Desmethyl Metabolite”, J. Clin. Pharmacol. 32:716-724 (1992).

O-desmethylvenlafaxine and processes for the preparation thereof are described in U.S. Pat. Nos. 6,197,828 and 6,689,912, and in US 2005/0197392, which are incorporated herein by reference.

Venlafaxine base can be used as a starting material in the preparation of O-desmethylvenlafaxine, as demonstrated in U.S. Pat. No. 6,689,912, U.S. Pat. No. 6,197,828, WO 03/048104 and US 2005/0197392.

Several pharmaceutically acceptable salts of O-desmethylvenlafaxine are described in U.S. Pat. No. 4,535,186. In Example 26, the fumarate salt is prepared. The product is reported to have a melting point range of 140° C.-142° C. In Example 27, the hydrochloride salt is prepared. The product is reported to have a melting point range of 162° C.-164° C.

O-desmethylvenlafaxine succinate, its polymorphs, including forms I, II, III, and IV, and processes for preparation thereof are described in U.S. Pat. No. 6,673,838. Crystalline forms of O-desmethylvenlafaxine succinate are also disclosed in WO 2008/047167 and WO 2008/017886.

The discovery of new solid states of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional processes for preparation of pharmaceutically acceptable salts of O-desmethylvenlafaxine either in a crystalline or in an amorphous form.

### SUMMARY OF THE INVENTION

In one embodiment the present invention provides a crystalline O-desmethylvenlafaxine oxalate, characterized by data selected from the group consisting of: a PXRD pattern with peaks at about 5.2, 10.4, and 26.4±0.2 degrees 2-theta and at least two peaks selected from the following list of peaks at about: 11.6, 14.4, 16.8 and 19.2±0.2 degrees 2-theta; a PXRD pattern with peaks at about 14.4, 16.8 and 19.2±0.2 degrees 2-theta and at least two peaks selected from the following list of peaks at about: 5.2, 10.4 11.6, 13.1 and 26.4±0.2 degrees 2-theta; a PXRD pattern with peaks at about 11.6, 13.1, 14.4, 16.8 and 19.2±0.2 degrees 2-theta; a powder XRD pattern substantially as depicted in FIG. 16; and combinations thereof.

In one embodiment the present invention provides a crystalline Form I of O-desmethylvenlafaxine hydrochloride characterized by data selected from the group consisting of: a PXRD pattern with peaks at about 5.7, 11.5, 17.3, 19.1 and 23.1±0.2 degrees 2-theta; a PXRD pattern as depicted in FIG. 15; and combinations thereof.

In one embodiment the present invention provides a crystalline Form II of O-desmethylvenlafaxine hydrochloride, characterized by data selected from the group consisting of: a PXRD pattern with peaks at about 10.2, 13.2 and 16.6±0.2 degrees 2-theta, and at least two peaks selected from the following list of peaks at about: 19.2, 25.9, 27.3 and 31.7±0.2 degrees 2-theta; a PXRD pattern with peaks at about 10.2, 13.2, 16.6, 25.9 and 31.7±0.2 degrees 2-theta; a PXRD pattern as depicted in FIG. 17; and combinations thereof.

In one embodiment the present invention provides a crystalline Form III of O-desmethylvenlafaxine hydrochloride, characterized by data selected from the group consisting of: a PXRD pattern with peaks at about 12.1, 13.1 and 14.6±0.2 degrees 2-theta, and at least two peaks selected from the following list of peaks at about: 5.9, 16.8, 18.8, 20.5 and 21.2±0.2 degrees 2-theta; a PXRD pattern with peaks at about 12.1, 13.1, 14.6, 18.8 and 20.5±0.2 degrees 2-theta; a PXRD pattern as depicted in FIG. 18; and combinations thereof.

In one embodiment the present invention provides a process for the preparation of ODV-succinate salt characterized by an X-ray powder diffraction pattern having characteristic peaks at 10.20, 14.91, 20.56, 22.13, 23.71, 24.60, and 25.79 degrees 2 theta±0.2 degrees 2 theta (Form I) comprising: a) providing a mixture of O-desmethylvenlafaxine, succinic acid, C₁-C₄ alcohol and water; b) heating the mixture to obtain a solution; c) cooling the solution to obtain a suspension of ODV-succinate; d) heating the suspension to a temperature of about 50° C. to about 60° C.; and e) cooling the suspension to obtain the crystalline form of ODV-succinate (Form I).

In one embodiment the present invention provides amorphous forms of the following salts of O-desmethylvenlafaxine: a hydrochloride salt, a sulfuric acid salt, a citrate salt, a maleate salt, a mesylate salt, a mandelate salt, a malic acid salt, a quinic acid salt a tartrate salt, and a palmitate salt.

### BRIEF DESCRIPTION OF THE FIGURES

FIGS. 1 and 2 represent a powder XRD pattern of an amorphous form of O-desmethylvenlafaxine hydrochloride salt.

FIGS. 3 and 4 represent a powder XRD pattern of an amorphous form of O-desmethylvenlafaxine sulfuric acid salt.

FIGS. 5 and 10 represent a powder XRD pattern of an amorphous form of O-desmethylvenlafaxine mesylate salt.

FIG. 6 represents a powder XRD pattern of an amorphous form of O-desmethylvenlafaxine citrate salt.

FIG. 7 represents a powder XRD pattern of a pure amorphous form of O-desmethylvenlafaxine citrate salt.

FIGS. 8 and 9 represent a powder XRD pattern of an amorphous form of O-desmethylvenlafaxine maleate salt.

FIG. 11 represents a powder XRD pattern of an amorphous form of O-desmethylvenlafaxine mandelate salt.

FIG. 12 represents a powder XRD pattern of an amorphous form of O-desmethylvenlafaxine malic acid salt.

FIG. 13 represents a powder XRD pattern of an amorphous form of O-desmethylvenlafaxine quinic acid salt.

FIG. 14 represents a powder XRD pattern of an amorphous form of O-desmethylvenlafaxine tartrate salt.

FIG. 15 represents a powder XRD pattern of a crystalline form I of O-desmethylvenlafaxine hydrochloride salt.

FIG. 16 represents a powder XRD pattern of a crystalline form of O-desmethylvenlafaxine oxalate salt.

FIG. 17 represents a powder XRD pattern of a crystalline form II of O-desmethylvenlafaxine hydrochloride salt.

FIG. 18 represents a powder XRD pattern of a crystalline form III of O-desmethylvenlafaxine hydrochloride salt.

### DETAILED DESCRIPTION OF THE INVENTION

As used herein, the term “room temperature” refers to a temperature of about 20° C. to about 35° C., more preferably about 20° C. to about 25° C. and most preferably about 25° C.

As used here, the term “under reduced pressure” refers to a pressure of less than 100 mm Hg, more preferably less than 50 mm Hg, most preferably less than 10 mm Hg. For example, between about 2 mm Hg and about 20 mm Hg or between about 5 mm Hg and about 8 mm Hg.

As used here, the term “pure” when used in respect of amorphous forms refers to having less than 5% (w/w) of crystalline, preferably less than 2% (w/w) of crystalline, more preferably less than 1% (w/w) of any crystalline amount in the O-desmethylvenlafaxine salt. The presence of a particular crystalline O-desmethylvenlafaxine can be determined by the presence of PXRD peaks characteristic of crystalline forms of O-desmethylvenlafaxine salts. The amount of crystallinity is quantified by methods known in the art like “crystallinity index” available to most XRD software.

In one embodiment, the invention provides a crystalline O-desmethylvenlafaxine oxalate, characterized by data selected from the group consisting of: a PXRD pattern with peaks at about 5.2, 10.4, and 26.4±0.2 degrees 2-theta and at least two peaks selected from the following list of peaks at about: 11.6, 14.4, 16.8 and 19.2±0.2 degrees 2-theta; a PXRD pattern with peaks at about 14.4, 16.8 and 19.2±0.2 degrees 2-theta and at least two peaks selected from the following list of peaks at about: 5.2, 10.4 11.6, 13.1 and 26.4±0.2 degrees 2-theta; a PXRD pattern with peaks at about 11.6, 13.1, 14.4, 16.8 and 19.2±0.2 degrees 2-theta; a powder XRD pattern substantially as depicted in FIG. 16; and combinations thereof.

The crystalline form of O-desmethylvenlafaxine oxalate salt may be prepared by a process comprising suspending O-desmethylvenlafaxine base characterized by X-ray powder diffraction reflections at about 12.1, 13.2, 15.9 and 20.4±0.2 degrees two theta, oxalic acid and a C₄-C₈ ether to obtain a suspension. O-desmethylvenlafaxine oxalate salt is then recovered out of the mixture.

The suspension is preferably maintained at room temperature for about 4 hours to about 24 hours, preferably about 12 hours.

Preferably, the solvent is cyclopentyl methyl ether.

The oxalic acid can be in its di-hydrate form.

The resulting precipitate may be recovered by conventional techniques, such as filtration. The precipitate may be dried under ambient conditions or reduced pressure, or elevated temperature. Preferably, for about 4 hours to about 24 hours, more preferably about 12 hours. In one embodiment, the precipitate is dried at room temperature at a pressure of less than about 100 mmHg.

The present invention further provides a process for the preparation of a mixture of an amorphous form of O-desmethylvenlafaxine oxalate salt and a crystalline form of O-desmethylvenlafaxine oxalate salt,characterized by data selected from the group consisting of: a PXRD pattern with peaks at about 5.2, 10.4, and 26.4±0.2 degrees 2-theta and at least two peaks selected from the following list of peaks at about:11.6, 14.4, 16.8 and 19.2±0.2 degrees 2-theta; and a PXRD pattern with peaks at about 14.4, 16.8 and 19.2±0.2 degrees 2-theta and at least two peaks selected from the following list of peaks at about: 5.2, 10.4, 11.6, 13.1 and 26.4±0.2 degrees 2-theta. This process comprises combining O-desmethylvenlafaxine, oxalic acid and a C₁-C₄ alcohol and removing of the solvent, preferably by evaporation to dryness the above mixture of the amorphous form and the crystalline form.

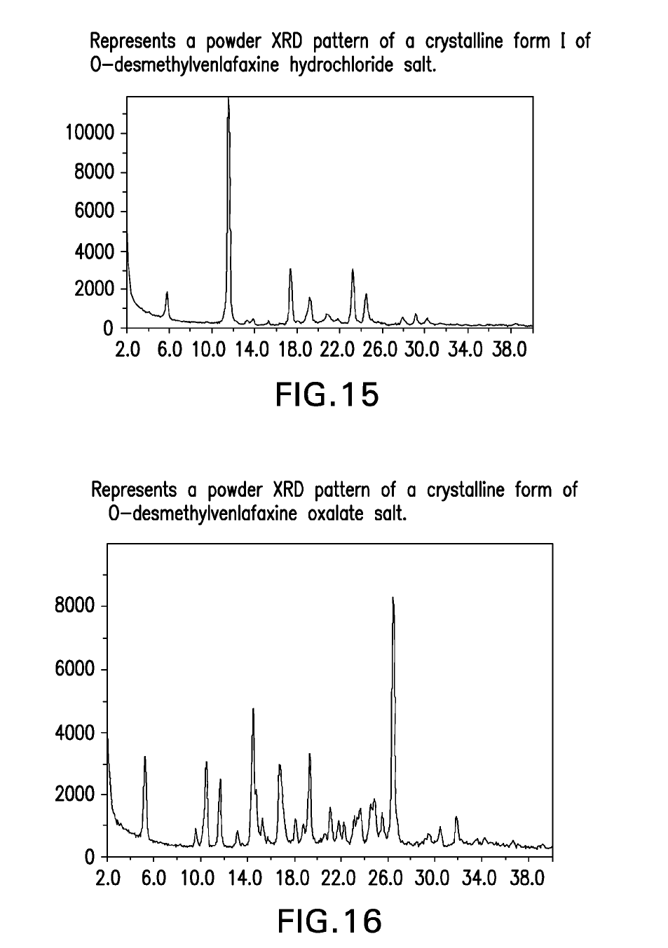

In another embodiment, the invention provides a crystalline form of O-desmethylvenlafaxine hydrochloride salt, characterized by data selected from the group consisting of: a PXRD pattern with peaks at about 5.7, 11.5, 17.3, 19.1 and 23.1±0.2 degrees 2-theta; a PXRD pattern as depicted in FIG. 15; and combinations thereof. This form can be denominated O-desmethylvenlafaxine hydrochloride salt crystalline Form I.

O-desmethylvenlafaxine hydrochloride salt Form I may be prepared by a process comprising suspending O-desmethylvenlafaxine base characterized by X-ray powder diffraction reflections at about 12.1, 13.2, 15.9, and 20.4±0.2 degrees two theta, hydrochloric acid and a C₄-C₈ ether to obtain a suspension. O-desmethylvenlafaxine hydrochloride salt Form I is then recovered out of the mixture.

The suspension is preferably maintained at room temperature for about 4 hours to about 24 hours, preferably about 12 hours.

Preferably, the solvent is cyclopentyl methyl ether.

The hydrochloric acid can be in a mixture with an alcohol, such as isopropanol.

The resulting precipitate may be recovered by conventional techniques, such as filtration. The precipitate may be dried under ambient conditions or reduced pressure, or elevated temperature. Preferably, for about 4 hours to about 24 hours, more preferably about 12 hours. In one embodiment, the precipitate is dried at room temperature at a pressure of less than about 100 mmHg.

The O-desmethylvenlafaxine base characterized by X-ray powder diffraction reflections at about 12.1, 13.2, 15.9, and 20.4±0.2 degrees two theta can be obtained by any method known to the skilled artisan, such as described in PCT publication WO2007120925 and US publication 2009/0137846, e.g., demethylating didesmethylvenlafaxine to obtain tridesmethylvenlafaxine in a reaction mixture; and converting the tridesmethyl venlafaxine to O-desmethylvenlafaxine without recovering the tridesmethyl venlafaxine from the reaction mixture.

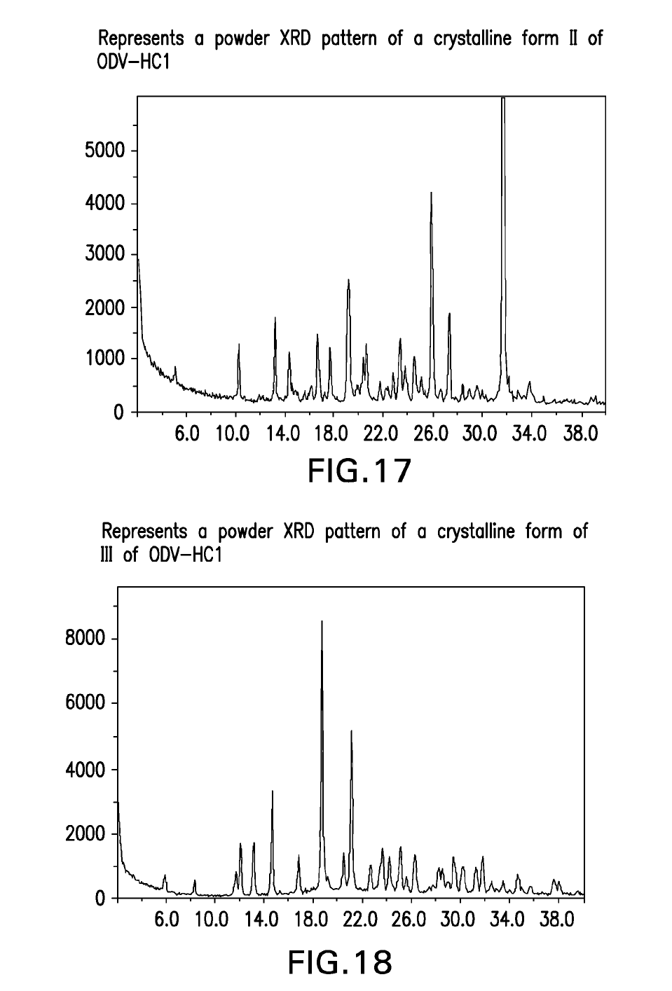

In another embodiment, the invention provides a crystalline form of O-desmethylvenlafaxine hydrochloride salt, characterized by data selected from the group of: a PXRD pattern with peaks at about 10.2, 13.2 and 16.6±0.2 degrees 2-theta, and at least two peaks selected from the following list of peaks at about: 19.2, 25.9, 27.3 and 31.7±0.2 degrees 2-theta; a PXRD pattern with peaks at about 10.2, 13.2, 16.6, 25.9 and 31.7±0.2 degrees 2-theta; a PXRD pattern as depicted in FIG. 17; and combinations thereof. This form can be denominated O-desmethylvenlafaxine hydrochloride salt crystalline Form II.

O-desmethylvenlafaxine hydrochloride salt Form II may be prepared by a process comprising suspending amorphous O-desmethylvenlafaxine hydrochloride, in a mixture of a C₄-C₈ ether and at least two solvents from the group of C₁-C₄ alcohols to obtain a suspension. O-desmethylvenlafaxine hydrochloride salt Form II is then recovered out of the mixture.

The amorphous O-desmethylvenlafaxine hydrochloride starting material can be prepared as described below.

The suspension is preferably maintained at room temperature for about 4 hours to about 24 hours, preferably about 12 hours.

Preferably, the ratio of solvents used is between about 3:1 and about 1:3, preferably about 2:1 alcohol to ether.

Preferably, the mixture of C₁-C₄ alcohols consists of methanol and isopropanol (IPA).

Preferably, the ether used is cyclopentyl methyl ether.

In one specific embodiment, amorphous O-desmethylvenlafaxine hydrochloride is suspended in a mixture of methanol, isopropanol and cyclopentyl methyl ether to obtain O-desmethylvenlafaxine hydrochloride salt Form II. Preferably the suspension is in room temperature.

The resulting precipitate may be recovered by conventional techniques, such as filtration. The precipitate may be dried under ambient conditions or reduced pressure, or elevated temperature. Preferably, for about 4 hours to about 24 hours, more preferably about 12 hours. In one embodiment, the precipitate is dried at room temperature at a pressure of less than about 100 mmHg.

In another embodiment, the invention provides a crystalline form of O-desmethylvenlafaxine hydrochloride salt, characterized by data selected from to group of: a PXRD pattern with peaks at about 12.1, 13.1 and 14.6±0.2 degrees 2-theta, and at least two peaks selected from the following list of peaks at about:5.9, 16.8, 18.8, 20.5 and 21.2±0.2 degrees 2-theta; a PXRD pattern with peaks at about 12.1, 13.1, 14.6, 18.8 and 20.5±0.2 degrees 2-theta; a PXRD pattern as depicted in FIG. 18; and combinations thereof. This form can be denominated O-desmethylvenlafaxine hydrochloride salt crystalline Form III.

O-desmethylvenlafaxine hydrochloride salt Form III may be prepared by a process comprising suspending amorphous O-desmethylvenlafaxine hydrochloride, in a C₁-C₄ alcohol and a C₄-C₈ ether. O-desmethylvenlafaxine hydrochloride salt Form III is then recovered out of the mixture.

The suspension is preferably maintained at room temperature for about 4 hours to about 24 hours, preferably about 12 hours.

Preferably, the ratio of solvents used is between about 1:1 and about 3:1, preferably about 2:1 ether to alcohol.

Preferably, the ether used is cyclopentyl methyl ether and the alcohol used is isopropanol.

The resulting precipitate may be recovered by conventional techniques, such as filtration. The precipitate may be dried under ambient conditions or reduced pressure, or elevated temperature. Preferably, for about 4 hours to about 24 hours, more preferably about 12 hours. In one embodiment, the precipitate is dried at room temperature at a pressure of less than about 100 mmHg.

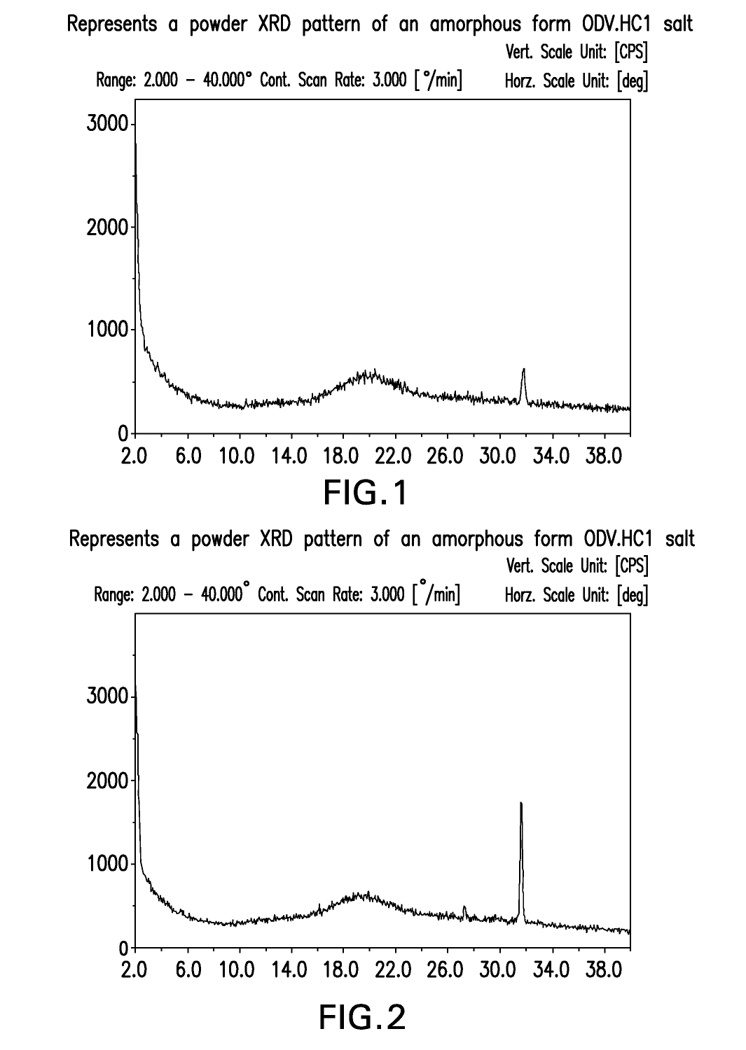

In another embodiment, the invention provides amorphous O-desmethylvenlafaxine hydrochloride salt, as depicted in FIGS. 1 and 2.

The amorphous O-desmethylvenlafaxine hydrochloride salt may be prepared by a process comprising providing a solution of O-desmethylvenlafaxine, hydrochloric acid and C₁-C₄ alcohol and removing the solvent to obtain a precipitate.

The solution can be obtained at room temperature up to the reflux temperature of the solvent.

The O-desmethylvenlafaxine starting material is preferably in its base form.

Preferably, the solvent is ethanol or isopropanol.

To obtain the solid, the solvent is preferably removed by evaporation, more preferably under reduced pressure. Optionally, prior to the removal of solvent, the solution is cooled to less than about 10° C., preferably less than about 0° C., more preferably less than about −5° C., for example about −10° C. Optionally, prior to the removal of the solvent, the solution is combined with methyl isobutyl ketone (MIBK). Preferably, the solution is cooled prior to the addition of MIBK.

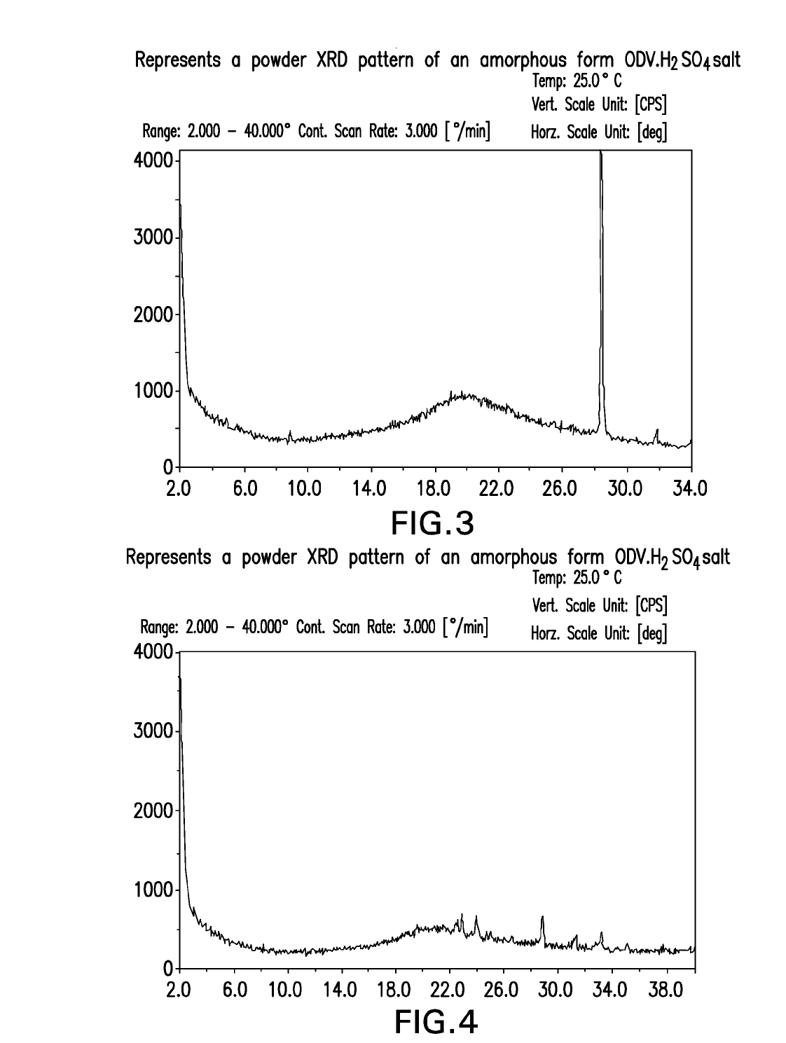

In another embodiment, the invention provides amorphous O-desmethylvenlafaxine sulfuric acid salt, as depicted in FIGS. 3 and 4.

The amorphous O-desmethylvenlafaxine sulfuric acid salt may be prepared by a process comprising combining O-desmethylvenlafaxine, sulfuric acid and a solvent selected from a C₁-C₄ alcohol and a C₃-C₈ ketone and removing the solvent to obtain O-desmethylvenlafaxine sulfuric acid.

The solution can be obtained at room temperature up to the reflux temperature of the solvent.

The O-desmethylvenlafaxine starting material is preferably in its base form.

Preferably, the solvent is ethanol or acetone.

To obtain the solid, the solvent is preferably removed by evaporation, more preferably under reduced pressure.

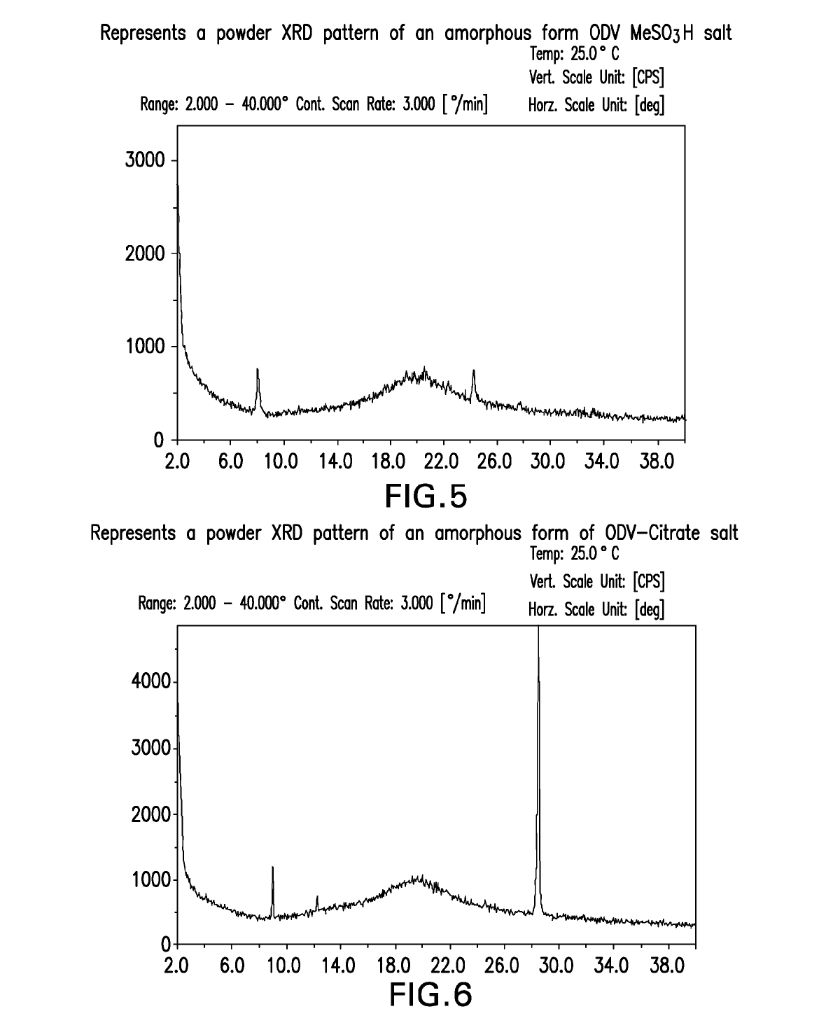

In another embodiment, the invention provides amorphous O-desmethylvenlafaxine mesylate salt, as depicted in FIGS. 5 and 10.

The amorphous O-desmethylvenlafaxine mesylate salt may be prepared by a process comprising combining O-desmethylvenlafaxine, methanesulfonic acid and a C₁-C₄ alcohol and removing the solvent to obtain O-desmethylvenlafaxine mesylate.

The solution can be obtained at room temperature up to the reflux temperature of the solvent.

The O-desmethylvenlafaxine starting material is preferably in its base form.

Preferably, the solvent is methanol or ethanol.

To obtain the solid, the solvent is preferably removed by evaporation, more preferably under reduced pressure.

In another embodiment, the invention provides amorphous O-desmethylvenlafaxine citrate salt, as depicted in FIG. 6.

The amorphous O-desmethylvenlafaxine citrate salt may be prepared by a process comprising providing a solution of O-desmethylvenlafaxine, citric acid and ethanol and removing the solvent to obtain a precipitate.

Typically, the solution is obtained at room temperature.

Preferably, the ethanol used, is an absolute ethanol.

The O-desmethylvenlafaxine starting material is preferably in its base form.

To obtain the solid, the solvent is preferably removed by evaporation, more preferably under reduced pressure.

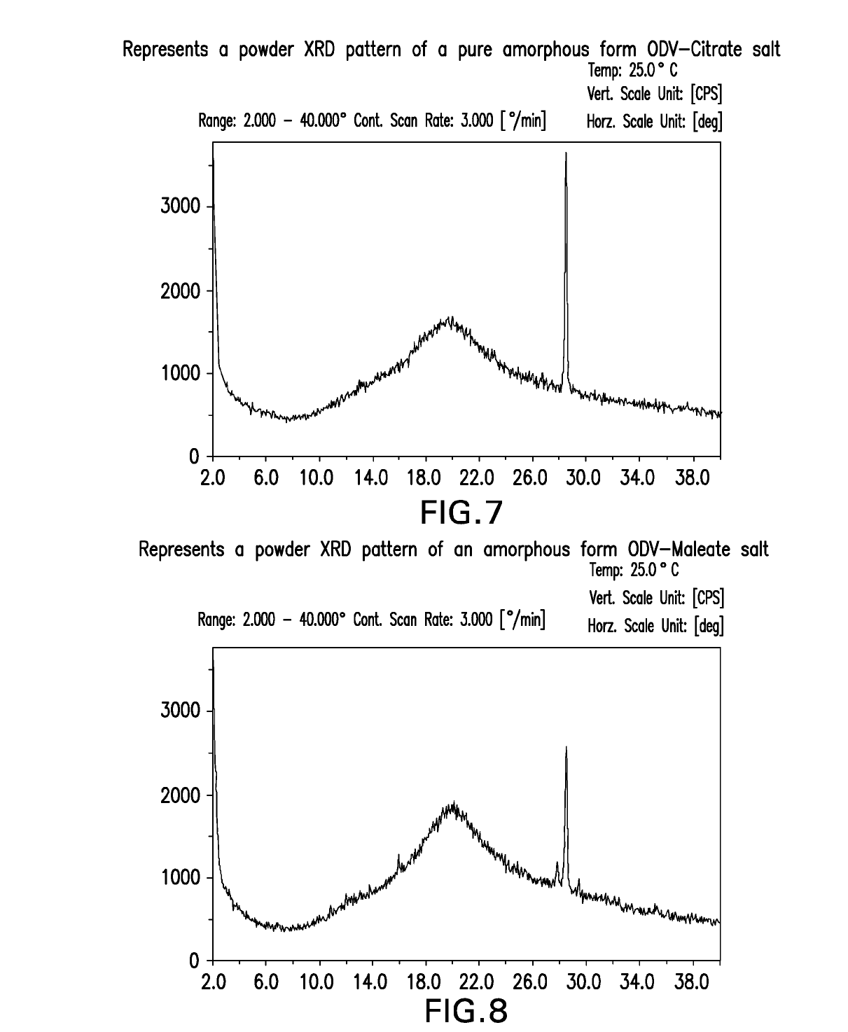

In another embodiment, the invention provides pure amorphous O-desmethylvenlafaxine citrate salt, as depicted in FIG. 7.

The pure amorphous O-desmethylvenlafaxine citrate salt may be prepared by a process comprising providing a solution of O-desmethylvenlafaxine, citric acid and methanol and removing the solvent to obtain a precipitate.

Typically, the solution is obtained at reflux temperature.

The O-desmethylvenlafaxine starting material is preferably in its base form.

To obtain the solid, the solvent is preferably removed by evaporation, more preferably under reduced pressure.

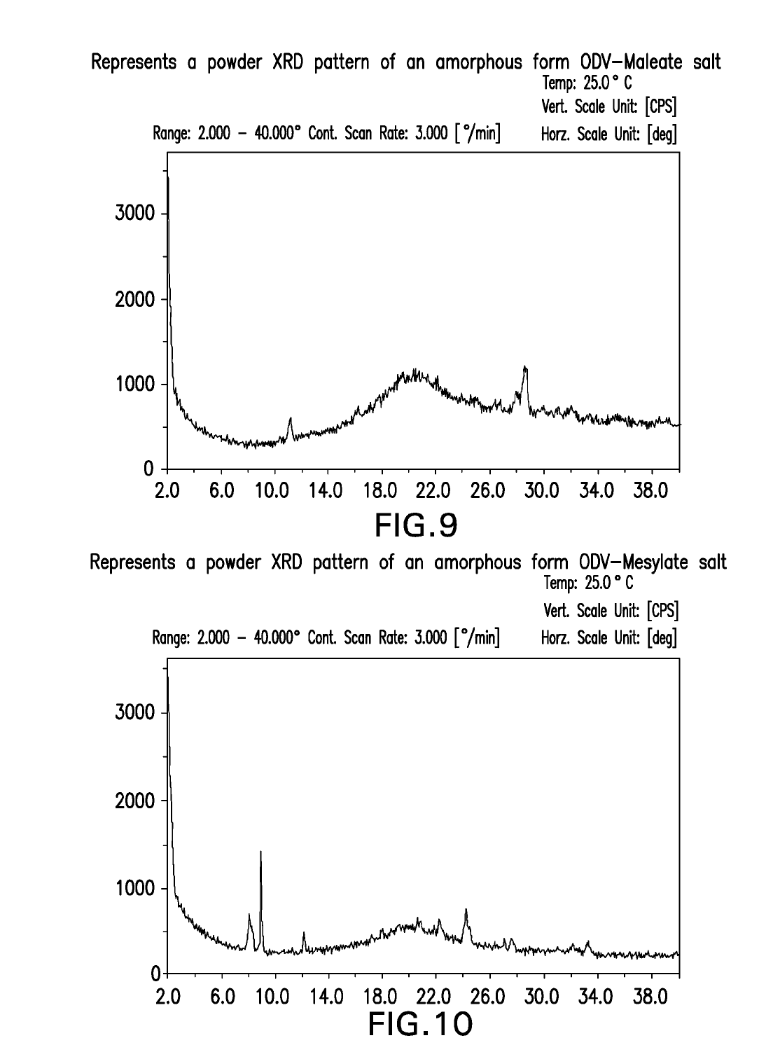

In another embodiment, the invention provides amorphous O-desmethylvenlafaxine maleate salt, as depicted in FIGS. 8 and 9.

The amorphous O-desmethylvenlafaxine maleate salt may be prepared by a process comprising combining O-desmethylvenlafaxine, maleic acid and a solvent selected from a C₁-C₄ alcohol and a C₃-C₈ ether and removing the solvent to obtain O-desmethylvenlafaxine maleate.

The solution can be obtained at room temperature up to the reflux temperature of the solvent.

The O-desmethylvenlafaxine starting material is preferably in its base form.

Preferably, the solvent is ethanol or methyl tent-butyl ether.

To obtain the solid, the solvent is preferably removed by evaporation, more preferably under reduced pressure.

In another embodiment, the invention provides amorphous O-desmethylvenlafaxine mandelate salt as depicted in FIG. 11.

The amorphous O-desmethylvenlafaxine mandelate salt may be prepared by a process comprising providing a solution of O-desmethylvenlafaxine, mandelic acid and a C₁-C₄ alcohol and removing the solvent to obtain a precipitate.

The solution can be obtained at room temperature up to the reflux temperature of the solvent.

The O-desmethylvenlafaxine starting material is preferably in its base form.

Optionally, the mandelic acid is D-mandelic acid.

Preferably, the solvent is ethanol.

To obtain the solid, the solvent is preferably removed by evaporation, more preferably under reduced pressure. Optionally, prior to the removal of solvent, the solution is cooled to less than about 10° C., preferably less than about 0° C., more preferably less than about −5° C., for example about −10° C. Optionally, prior to the removal of the solvent, the solution is combined with heptane. Preferably, the solution is cooled prior to the addition of heptane.

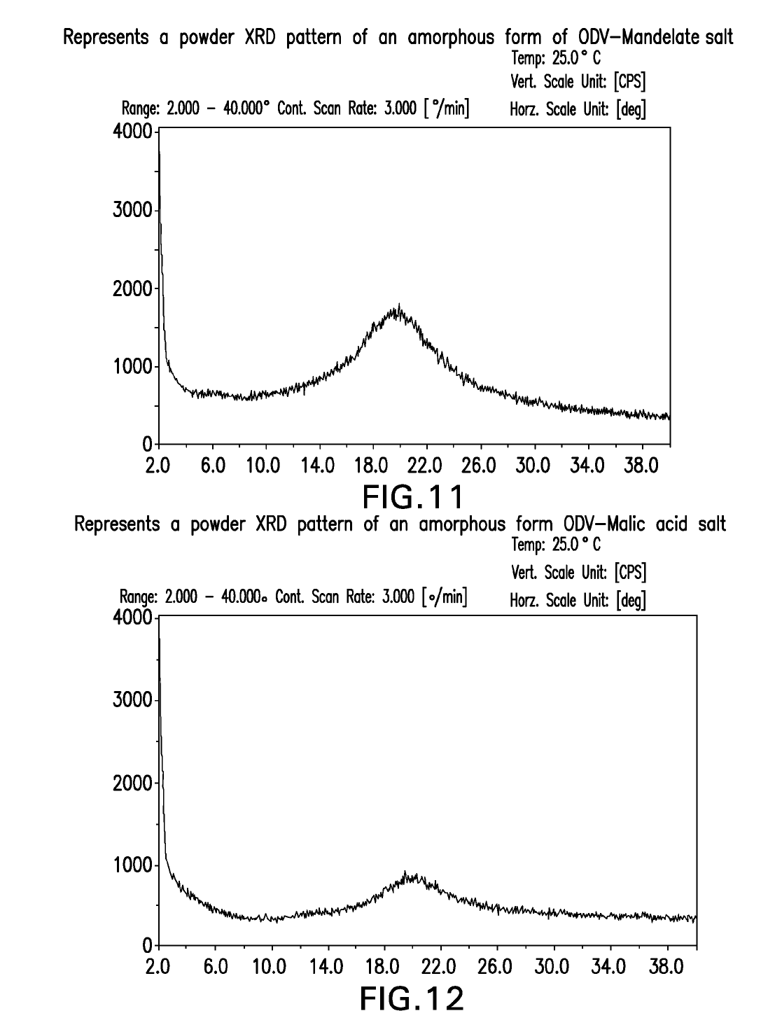

In another embodiment, the invention provides amorphous O-desmethylvenlafaxine malic acid salt, as depicted in FIG. 12.

The amorphous O-desmethylvenlafaxine malic acid salt may be prepared by a process comprising providing a solution of O-desmethylvenlafaxine, malic acid and C₁-C₄ alcohol and removing the solvent to obtain a precipitate.

Typically, the solution is obtained at reflux temperature.

The O-desmethylvenlafaxine starting material is preferably in its base form.

Optionally, the malic acid is D-malic acid.

Preferably, the C₁-C₄ alcohol is ethanol.

Optionally, prior to the removal of solvent, the solution is cooled to less than about 10° C., preferably less than about 0° C., more preferably less than about −5° C., for example about −10° C. Optionally, prior to the removal of the solvent, the solution is combined with cyclohexanane. Preferably, the solution is cooled prior to the addition of cyclohexanane.

To obtain the solid, the solvent is preferably removed by evaporation, more preferably under reduced pressure.

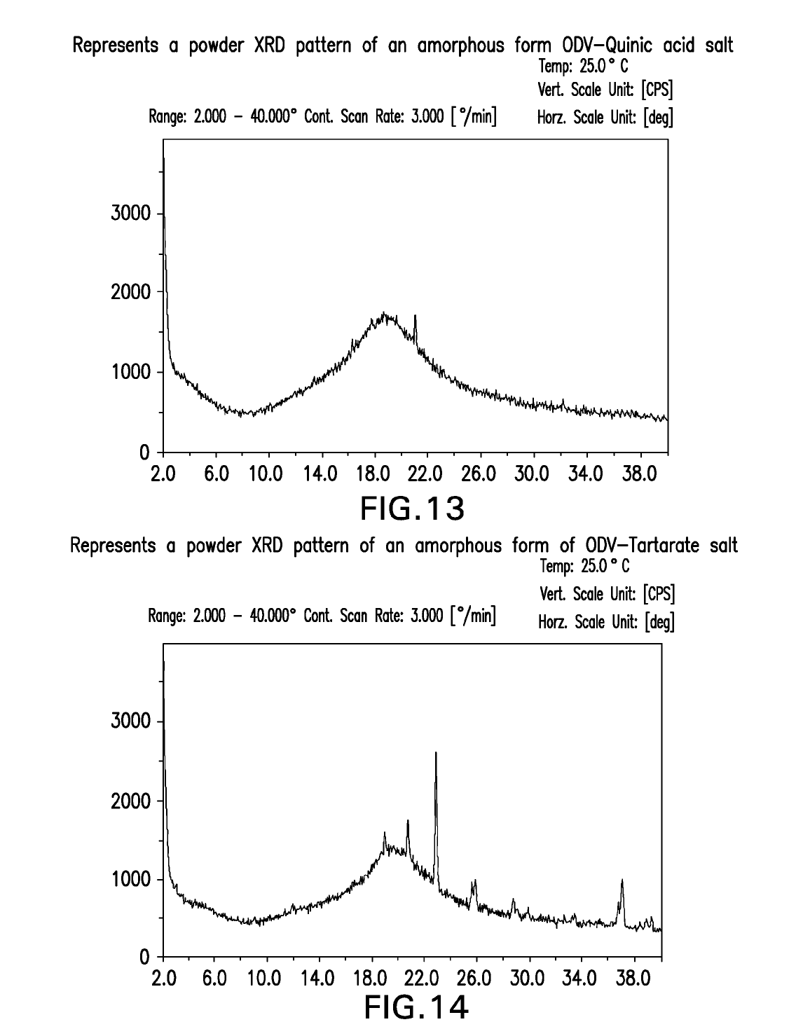

In another embodiment, the invention provides amorphous O-desmethylvenlafaxine quinic acid salt, as depicted in FIG. 13.

The amorphous O-desmethylvenlafaxine quinic acid salt may be prepared by a process comprising providing a solution of O-desmethylvenlafaxine, quinic acid and a C₁-C₄ alcohol and removing the solvent to obtain a precipitate.

The solution can be obtained at room temperature or at the reflux temperature of the solvent.

The O-desmethylvenlafaxine starting material is preferably in its base form.

Optionally, the quinic acid is D-quinic acid.

Preferably, the solvent is ethanol.

To obtain the solid, the solvent is preferably removed by evaporation, more preferably under reduced pressure. Optionally, prior to the removal of solvent, the solution is cooled to less than about 10° C., preferably less than about 0° C., more preferably less than about −5° C., for example about −10° C. Optionally, prior to the removal of the solvent, the solution is combined with toluene. Preferably, the solution is cooled prior to the addition of toluene.

In another embodiment, the invention provides amorphous O-desmethylvenlafaxine tartrate salt, as depicted in FIG. 14.

The amorphous O-desmethylvenlafaxine tartrate salt may be prepared by a process comprising providing a solution of O-desmethylvenlafaxine, tartaric acid and a C₁-C₄ alcohol and removing the solvent to obtain a precipitate.

The solution can be obtained at room temperature or at the reflux temperature of the solvent.

The O-desmethylvenlafaxine starting material is preferably in its base form.

Optionally, the tartaric acid is L-tartaric acid.

Preferably, the solvent is ethanol.

To obtain the solid, the solvent is preferably removed by evaporation, more preferably under reduced pressure. Optionally, prior to the removal of solvent, the solution is cooled to less than about 10° C., preferably less than about 0° C., more preferably less than about −5° C., for example about −10° C. Optionally, prior to the removal of the solvent, the solution is combined with toluene. Preferably, the solution is cooled prior to the addition of toluene.

In another embodiment, the invention provides a process for preparing crystalline form of O-desmethylvenlafaxine succinate (ODV-succinate) salt characterized by an X-ray powder diffraction pattern having characteristic peaks at 10.20, 14.91, 20.56, 22.13, 23.71, 24.60, and 25.79 degrees 2 theta±0.2 degrees 2 theta (Form I) comprising a) providing a mixture of O-desmethylvenlafaxine, succinic acid, C₁-C₄ alcohol and water; b) heating the mixture to reflux to obtain a solution; c) cooling the solution to a temperature of about 0° C. to about room temperature to obtain a suspension of ODV-succinate; d) heating the suspension to a temperature of about 50° C. to about 60° C.; and e) cooling the suspension to obtain the crystalline form of ODV-succinate.

A skilled in the art would understand that the process of heating the mixture containing a precipitate of ODV-succinate and further cooling the suspension, can be repeated several times.

Preferably, the temperature of step c) is about 5 to about 15° C. Preferably, the cooling occurs over a period of about 1 to about 6 hours, preferably about 2 to about 5 hours, most preferably about 3 to about 4.5 hours.

Preferably, the mixture in step d) is heated for about 2 to about 10 hours. More preferably, for about 4 to about 6 hours. Preferably, the mixture in step d) is heated to temperature of about 55 to about 60° C. for about 2 to about 10 hours. More preferably, for about 4 to about 6 hours.

Preferably, the mixture in step e) is cooled to a temperature of about 0° C. to about room temperature. More preferably to a temperature of about 0° C. to about 10° C.

Preferably, the mixture in step e) is stirred for about 4 to about 30 hours, for example about 24 hours. More preferably, for about 10 to 16 hours. Most preferably, for about 12 hours.

The resulting precipitate may be recovered by conventional techniques, such as filtration. The precipitate may be dried under ambient conditions or reduced pressure, or elevated temperature.

Preferably, the solvent is isopropanol.

Preferably, the O-desmethylvenlafaxine starting material is in its base form.

The present invention further encompasses 1) a pharmaceutical composition comprising any one, or combination, of the crystalline Forms and/or amorphous form described above and at least one pharmaceutically acceptable excipient and 2) the use of any one, or combination, of the above-described crystalline Forms and/or amorphous form, in the manufacture of a pharmaceutical composition, wherein the pharmaceutical composition can be useful for the treatment of depression.

The pharmaceutical composition of the present invention can be in a solid or a non-solid form. If the pharmaceutical composition is in a non-solid form, any one, or combination, of the crystalline Forms and/or amorphous within the composition, are retained as solid(s) in the non-solid pharmaceutical composition, e.g., as a suspension, foam, ointment and etc.

The pharmaceutical composition can be prepared by a process comprising combining any one, or combination, of the above-described crystalline Forms and/or amorphous form with at least one pharmaceutically acceptable excipient. The crystalline Forms and/or amorphous form can be obtained by any of the processes of the present invention as described above.

The pharmaceutical composition can be used to make appropriate dosage forms such as tablets, powders, capsules, suppositories, sachets, troches and losenges.

Any one, or combination, of the above-described crystalline Forms and/or amorphous form of the present invention, particularly in a pharmaceutical composition and dosage form, can be used to treat depression in a mammal such as a human, comprising administering a treatment effective amount of the one, or combination, of the crystalline Forms and/or amorphous form in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to limit its scope in any way.

### EXAMPLES

The x-ray powder diffraction was performed on an ARL X-ray powder diffractometer model X'TRA-019, Copper Kα₁ radiation (λ=1.5418 Å) was used. The sample holder was a round standard aluminum sample holder with round zero background plate (quartz).The scanning parameters were: range: 2-40 degrees two-theta; scan mode: continuous scan; step size: 0.05°; and scan rate: 3 degrees/minute.

### Preparation of ODV-HCl

### Example 1

A 100 ml flask equipped with a magnetic stirrer was charged with ODV base (10 g, 37.96 mmol) and isopropanol (IPA) (100 ml), the suspension being stirred at room temperature. HCl/IPA (3 ml, 36.19 mmol) was added at room temperature and a clear solution was obtained. After overnight stirring, the solution was evaporated to dryness in order to get amorphous ODV.HCl salt. X-ray powder diffraction pattern is depicted in FIG. 1.

### Example 2

A 100 ml flask equipped with a magnetic stirrer was charged with ODV base (3 g, 11.39 mmol) and EtOH 95% (9 ml), the suspension being stirred at reflux. HCl, 37% (1.23 g, 12.47 mmol) was added and a clear solution was obtained. After stirring overnight at RT, the mixture was cooled to −10° C. Methyl Isobutyl Ketone (MIBK) (6 ml) was added and a clear solution appeared. The mixture was evaporated to dryness to get amorphous ODV.HCl salt. X-ray powder diffraction pattern is depicted in FIG. 2.

### Preparation of ODV-H₂SO₄

### Example 3

A 100 ml flask equipped with a magnetic stirrer was charged with ODV base (10 g, 37.96 mmol) and acetone (100 ml), the suspension being stirred at room temperature. H₂SO₄ 98% (3.75 g, 38.26 mmol) was added at room temperature. A clear solution and sticky lump were obtained. After overnight stirring, the mixture was evaporated to dryness to get amorphous ODV.H₂SO₄ salt. X-ray powder diffraction pattern is depicted in FIG. 3.

### Example 4

A 100 ml flask equipped with a magnetic stirrer was charged with ODV base (3 g, 11.39 mmol) and EtOH 95% (15 ml), the suspension being stirred at reflux. H₂SO₄ 98% (1.2 g 12.24 mmol) was added and a clear solution was obtained. After stirring overnight at room temperature, the mixture was evaporated to dryness in order to get amorphous ODV.H₂SO₄. X-ray powder diffraction pattern is depicted in FIG. 4.

### Preparation of ODV-Citrate

### Example 5

A 100 ml flask equipped with a magnetic stirrer was charged with ODV base (6 g, 22.78 mmol) and EtOH abs (60 ml), the suspension being stirred at room temperature. Citric acid (3.37 g 17.54 mmol) was added at room temperature and a clear solution was obtained. After overnight stirring, the mixture was evaporated to dryness to get amorphous ODV Citrate. X-ray powder diffraction pattern is depicted in FIG. 6.

### Example 6

A 100 ml flask equipped with a magnetic stirrer was charged with ODV base (3 g, 11.39 mmol) and MeOH (15 ml), the suspension being stirred at reflux. Citric acid (2.4 g 12.49 mmol) was added and a clear solution was obtained. After overnight stirring, the mixture was evaporated to dryness in order to get amorphous ODV-Citrate salt. X-ray powder diffraction pattern is depicted in FIG. 7.

### Preparation of ODV-Maleate

### Example 7

A 100 ml flask equipped with a magnetic stirrer was charged with ODV base (6 g, 22.78 mmol) and Methyl Tertiary Butyl Ether (MTBE) (60 ml), the suspension being stirred at room temperature. Maleic acid (2.64 g 22.74 mmol) was added at room temperature and a clear solution and sticky lump were obtained. MeOH (30 ml) was added. After stirring overnight, the mixture was evaporated to dryness to get amorphous ODV-Maleic salt. X-ray powder diffraction pattern is depicted in FIG. 8.

### Example 8

A 100 ml flask equipped with a magnetic stirrer was charged with ODV base (3 g, 11.39 mmol) and EtOH 95% (15 ml), the suspension being stirred at reflux. Maleic acid (1.45 12.49 mmol) was added and a clear solution was obtained. After stirring overnight at room temperature, the mixture was evaporated to dryness in order to get amorphous ODV.Maleic salt. X-ray powder diffraction pattern is depicted in FIG. 9.

### Preparation of ODV-Mesylate

### Example 9

A 100 ml flask equipped with a magnetic stirrer was charged with ODV base (10 g, 37.96 mmol) and MeOH (100 ml), the suspension being stirred at room temperature. Methane sulfonic acid (2.5 ml 38.55 mmol) was added at room temperature. A clear solution and sticky lump were obtained. After overnight stirring, the mixture was evaporated to dryness to get amorphous ODV.Mesylate salt. X-ray powder diffraction pattern is depicted in FIG. 5.

### Example 10

A 100 ml flask equipped with a magnetic stirrer was charged with ODV base (3 g, 11.39 mmol) and EtOH 95% (15 ml), the suspension being stirred at reflux. Methane sulfonic acid (1.2 g 12.49 mmol) was added and a clear solution was obtained. After stirring overnight at room temperature, the mixture was evaporated to dryness in order to get amorphous ODV.Mesylate salt. X-ray powder diffraction pattern is depicted in FIG. 10.

### Preparation of ODV-Mandelate

### Example 11

A 100 ml one necked flask equipped with a magnetic stirrer was charged with ODV base (3 g, 11.39 mmol) and EtOH 95% (9 ml), the suspension being stirred at reflux. D-Mandelic acid (1.9 g 12.49 mmol) was added and a clear solution was obtained. After overnight stirring at room temperature, the mixture was cooled to −10° C. Heptane (6 ml) was added and a clear solution appeared. The mixture was evaporated to dryness to get amorphous ODV.Mandelate. X-ray powder diffraction pattern is depicted in FIG. 11.

### Preparation of ODV-Malic Acid

### Example 12

A 100 ml flask equipped with a magnetic stirrer was charged with ODV base (3 g, 11.39 mmol) and EtOH 95% (9 ml), the suspension being stirred at reflux. D-Malic acid (1.68 g 12.53 mmol) was added and a clear solution was obtained. After stirring overnight at room temperature, the mixture was cooled to −10° C. Cyclohexanane (6 ml) was added and a clear solution appeared. The mixture was evaporated to dryness to get amorphous ODV Malic. X-ray powder diffraction pattern is depicted in FIG. 12.

### Preparation of ODV-Quinic Acid

### Example 13

A 100 ml one necked flask equipped with a magnetic stirrer was charged with ODV base (3 g, 11.39 mmol) and EtOH 95% (9 ml), the suspension being stirred at reflux. D-Quinic acid (2.4 g 12.49 mmol) was added and a clear solution was obtained. After stirring overnight at room temperature, the mixture was cooled to −10° C. Toluene (6 ml) was added and a clear solution appeared. The mixture was evaporated to dryness to get amorphous ODV Quinic. X-ray powder diffraction pattern is depicted in FIG. 13.

### Preparation of ODV-Tartarate

### Example 14

A 100 ml flask equipped with a magnetic stirrer was charged with ODV base (3 g, 11.39 mmol) and EtOH 95% (9 ml), the suspension being stirred at reflux. L-Tartaric acid (2.0 g 12.57 mmol) was added and a clear solution was obtained. After stirring overnight at room temperature, the mixture was cooled to −10° C. Toluene (6 ml) was added and a clear solution appeared. The mixture was evaporated to dryness to get amorphous ODV Tartarate. X-ray powder diffraction pattern is depicted in FIG. 14.

### Preparation of ODV-Oxalate

### Example 15

A 100 ml flask equipped with a mechanical stirrer was charged with a crystalline form of ODV base characterized by PXRD pattern having peaks at about 12.1, 13.2, 15.9, and 20.4±0.2 degrees two theta (1 g 3.79 mmol), Oxalic acid di-hydrate (0.55 g 4.36 mmol), and cyclopentyl methyl ether (10 ml). This suspension was stirred at room temperature overnight.

The solid was filtered under reduced pressure, washed and dried at 50° C. under vacuum overnight to get ODV-Oxalate crystalline form. X-ray powder diffraction pattern is depicted in FIG. 16.

### Preparation of ODV-HCl Crystalline Form I

### Example 16

A 100 ml flask equipped with a mechanical stirrer was charged with ODV base characterized by PXRD pattern having peaks at about 12.1, 13.2, 15.9, and 20.4±0.2 degrees two theta (1 g 3.79 mmol), cyclopentyl methyl ether (10 ml), and 5N isopropanolic hydrochloride (1.15 ml 6.3 mmol). This suspension was stirred at room temperature overnight.

The solid was filtered under reduced pressure, washed and dried at 50° C. under vacuum overnight to get ODV-HCl crystalline form I. X-ray powder diffraction pattern is depicted in FIG. 15.

### Example 17

A 100 ml flask equipped with a mechanical stirrer was charged with ODV base characterized by PXRD pattern having peaks at about 12.1, 13.2, 15.9, and 20.4±0.2 degrees two theta (5 g 18.98 mmol) cyclopentyl methyl ether (50 ml) and 5N isopropanolic hydrochloride (6 ml 32.87 mmol).

This suspension was stirred at room temperature overnight. The solid was filtered under reduced pressure; washed and dried at 50° C. under vacuum overnight to get ODV-HCl crystalline form I.

### Preparation of ODV-HCl Crystalline Form II

### Example 18

Amorphous ODV HCl (2 g 6.7 mmol) was re-slurried with MeOH (3 ml) isopropanol (IPA) (3 ml) and cyclopentyl methyl ether (3 ml) at room temperature overnight.

The mixture was filtered and dried at 50° C. under vacuum to get ODV-HCl crystalline form II. X-ray powder diffraction pattern is depicted in FIG. 17.

### Preparation of ODV-HCl Crystalline Form III

### Example 19

A 100 ml flask equipped with a mechanical stirrer was charged with amorphous ODV HCl (10 g 33.34 mmol) cyclopentyl methyl ether (50 ml) and isopropanol (IPA) (25 ml).This suspension was stirred at room temperature overnight.

The solid was filtered under reduced pressure; washed and dried overnight at 50° C. under vacuum to get ODV-HCl crystalline form III. X-ray powder diffraction pattern is depicted in FIG. 18.

### Preparation of ODV-Oxalate—a Mixture of Amorphous and Crystalline Form

### Example 20

A 100 ml flask equipped with a magnetic stirrer was charged with ODV base (6 g, 22.78 mmol) and isopropanol (IPA) (60 ml), the suspension being stirred at room temperature. Oxalic acid (2.87 g 31.87 mmol) was added at room temperature a clear solution and sticky lump were obtained. MeOH (20 ml) was added and no precipitate appeared after overnight stirring. The mixture was then evaporated to dryness in order to get a mixture of amorphous and crystalline form of ODV Oxalate salt.

### Example 21

A 100 ml flask equipped with a magnetic stirrer was charged with ODV base (3 g, 11.39 mmol) and EtOH 95% (15 ml), the suspension being stirred at reflux. Oxalic acid×2H₂O (1.58 g 12.49 mmol) was added and a clear solution was obtained. After stirring overnight at room temperature, the mixture was evaporated to dryness in order to get a mixture of amorphous and crystalline form of ODV Oxalate salt.

### Preparation of ODV-Palmitate

### Example 22

A 100 ml one necked flask equipped with a magnetic stirrer was charged with ODV base (3 g, 11.39 mmol) and EtOH 95% (15 ml), the suspension being stirred at reflux. Palmitic acid (3 g 11.7 mmol) was added and a turbid solution appeared. A slurry was obtained after overnight stirring at room temperature. The mixture was filtered and dried at 50° C. under vacuum to get a mixture ODV base and ODV palmitate salt.

### Preparation of ODV-Succinate (Form I)

### Example 23

ODV base (100 g), isopropanol (IPA) (550 ml), Succinic acid (58.33 g) and water (200 ml) are charged into a stirred reactor. The mixture is heated to reflux (˜82° C.) and stirred 30 min to obtain full dissolution. Then the solution is filtered and re-heated to reflux to ensure full dissolution. The solution is then cooled to 5-15° C. and for precipitation of ODV succinate. The mixture stirred for 1.5-2 hrs and then heated to 55-60° C. The mixture is stirred at this temperature for 4-6 hrs, then gradually cooled to 0-10° C. during 3-4.5 hrs and then stirred for additional 12 hrs. ODV succinate is filtered and the wet cake is washed twice with IPA (100 ml each wash). The wet material is dried in a vacuum oven at <40° C.

1. A crystalline O-desmethylvenlafaxine oxalate, characterized by data selected from the group consisting of: a PXRD pattern with peaks at about 5.2, 10.4, and 26.4±0.2 degrees 2-theta and at least two peaks selected from the following list of peaks at about: 11.6, 14.4, 16.8 and 19.2±0.2 degrees 2-theta; a PXRD pattern with peaks at about 14.4, 16.8 and 19.2±0.2 degrees 2-theta and at least two peaks selected from the following list of peaks at about: 5.2, 10.4 11.6, 13.1 and 26.4±0.2 degrees 2-theta; a PXRD pattern with peaks at about 11.6, 13.1, 14.4, 16.8 and 19.2±0.2 degrees 2-theta; a powder XRD pattern as depicted in FIG. 16; and combinations thereof.

2. The crystalline form of claim 1, wherein the crystalline form is characterized by a PXRD pattern with peaks at about 14.4, 16.8 and 19.2±0.2 degrees 2-theta and at least two peaks selected from the group consisting of 5.2, 10.4, 11.6, 13.1 and 26.4±0.2 degrees 2-theta.

3. The crystalline form claim 1, wherein the crystalline form is characterized by a PXRD pattern with peaks at about 11.6, 13.1, 14.4, 16.8 and 19.2±0.2 degrees 2-theta.

4. A crystalline form of O-desmethylvenlafaxine hydrochloride (Form I) characterized by data selected from the group consisting of: a PXRD pattern with peaks at about 5.7, 11.5, 17.3, 19.1 and 23.1±0.2 degrees 2-theta; a PXRD pattern as depicted in FIG. 15; and combinations thereof.

5. The crystalline form of claim 4, wherein the crystalline form is characterized by a PXRD pattern with peaks at about 5.7, 11.5, 17.3, 19.1 and 23.1±0.2 degrees 2-theta.

6. A crystalline form of O-desmethylvenlafaxine hydrochloride (Form II) characterized by data selected from the group consisting of: a PXRD pattern with peaks at about 10.2, 13.2 and 16.6±0.2 degrees 2-theta, and at least two peaks selected from the following list of peaks at about: 19.2, 25.9, 27.3 and 31.7±0.2 degrees 2-theta; a PXRD pattern with peaks at about 10.2, 13.2, 16.6, 25.9 and 31.7±0.2 degrees 2-theta; a PXRD pattern as depicted in FIG. 17; and combinations thereof.

7. The crystalline form of claim 6, wherein the crystalline form is characterized by a PXRD pattern with peaks at about 10.2, 13.2, 16.6, 25.9 and 31.7±0.2 degrees 2-theta.

8. A crystalline form of O-desmethylvenlafaxine hydrochloride (Form III) characterized by data selected from the group consisting of: a PXRD pattern with peaks at about 12.1, 13.1 and 14.6±0.2 degrees 2-theta, and at least two peaks selected from the following list of peaks at about: 5.9, 16.8, 18.8, 20.5 and 21.2±0.2 degrees 2-theta; a PXRD pattern with peaks at about 12.1, 13.1, 14.6, 18.8 and 20.5±0.2 degrees 2-theta; a PXRD pattern as depicted in FIG. 18; and combinations thereof.

9. The crystalline form of claim 8, wherein the crystalline form is characterized by a PXRD pattern with peaks at about 12.1, 13.1, 14.6, 18.8 and 20.5±0.2 degrees 2-theta.

10. An amorphous O-desmethylvenlafaxine hydrochloride salt.

11. An amorphous O-desmethylvenlafaxine sulfuric acid salt.

12. An amorphous O-desmethylvenlafaxine citrate salt.

13. A pure amorphous O-desmethylvenlafaxine citrate salt.

14. An amorphous O-desmethylvenlafaxine maleate salt.

15. An amorphous O-desmethylvenlafaxine mesylate salt.

16. An amorphous O-desmethylvenlafaxine mandelate salt.

17. An amorphous O-desmethylvenlafaxine malic acid salt.

18. An amorphous O-desmethylvenlafaxine quinic acid salt.

19. An amorphous O-desmethylvenlafaxine tartrate salt.

20. A process for the preparation of ODV-succinate salt characterized by an X-ray powder diffraction pattern having characteristic peaks at 10.20, 14.91, 20.56, 22.13, 23.71, 24.60, and 25.79 degrees 2 theta±0.2 degrees 2 theta (Form I) comprising:
a) providing a mixture of O-desmethylvenlafaxine, succinic acid, C₁-C₄ alcohol and water;
b) heating the mixture to obtain a solution;
c) cooling the solution to obtain a suspension of ODV-succinate;
d) heating the suspension to a temperature of about 50° C. to about 60° C.; and
e) cooling the suspension to obtain the crystalline form of ODV-succinate (Form I).

21. The process of claim 20, wherein the O-desmethylvenlafaxine starting material is preferably in its base form.

22. The process of claims 20, wherein the C₁-C₄ alcohol is isopropanol.

23. The process of claim 20, wherein the heating temperature of step b) is a reflux temperature.

24. The process of claim 20, wherein the mixture in step c) is cooled to a temperature of about 0° C. to about room temperature.

25. The process of claim 24, wherein the mixture in step c) is cooled to a temperature of about 5° C. to about 15° C.

26. The process of claim 20, wherein the mixture in step d) is heated to a temperature of about 50° C. to about 60° C. for about 2 to about 10 hours.

27. The process of claim 26, wherein the mixture in step d) is heated to a temperature of about 55° C. to about 60° C. for about 4 to about 6 hours.

28. The process of claim 20, wherein the mixture in step e) is cooled to a temperature of about 0° C. to about room temperature.

29. The process of claim 28, wherein the mixture in step e) is cooled to a temperature of about 0° C. to about 10° C.

30. The process of claim 20, wherein the mixture in step e) is maintained for about 12 hours.

31. The process of claim 20, wherein steps d) and e) are repeated for several times.

32. A pharmaceutical composition comprising a compound selected from the group consisting of the crystalline O-desmethylvenlafaxine oxalate of claim 1, the crystalline form of O-desmethylvenlafaxine hydrochloride of claim 4, crystalline form of O-desmethylvenlafaxine hydrochloride of claim 6, crystalline form of O-desmethylvenlafaxine hydrochloride of claim 8, an amorphous O-desmethylvenlafaxine hydrochloride salt, an amorphous O-desmethylvenlafaxine sulfuric acid salt, an amorphous O-desmethylvenlafaxine citrate salt, an amorphous O-desmethylvenlafaxine maleate salt, an amorphous O-desmethylvenlafaxine mesylate salt, an amorphous O-desmethylvenlafaxine mandelate salt, an amorphous O-desmethylvenlafaxine malic acid salt, an amorphous O-desmethylvenlafaxine quinic acid salt, an amorphous O-desmethylvenlafaxine tartrate salt, and combinations thereof.

33. A process for preparing a pharmaceutical composition of claim 32 comprising combining a compound selected from the group consisting of the crystalline O-desmethylvenlafaxine oxalate of claim 1, the crystalline form of O-desmethylvenlafaxine hydrochloride of claim 4, crystalline form of O-desmethylvenlafaxine hydrochloride of claim 6, crystalline form of O-desmethylvenlafaxine hydrochloride of claim 8, an amorphous O-desmethylvenlafaxine hydrochloride salt, an amorphous O-desmethylvenlafaxine sulfuric acid salt, an amorphous O-desmethylvenlafaxine citrate salt, an amorphous O-desmethylvenlafaxine maleate salt, an amorphous O-desmethylvenlafaxine mesylate salt, an amorphous O-desmethylvenlafaxine mandelate salt, an amorphous O-desmethylvenlafaxine malic acid salt, an amorphous O-desmethylvenlafaxine quinic acid salt, an amorphous O-desmethylvenlafaxine tartrate salt, and combinations thereof with at least one pharmaceutically acceptable excipient.

34. (canceled)

35. (canceled)

36. A method of treating depression in a patient comprising administering to a patient suffering from depression a therapeutically effective amount of a compound selected from the group consisting of the crystalline O-desmethylvenlafaxine oxalate of claim 1, the crystalline form of O-desmethylvenlafaxine hydrochloride of claim 4, crystalline form of O-desmethylvenlafaxine hydrochloride of claim 6, crystalline form of O-desmethylvenlafaxine hydrochloride of claim 8, an amorphous O-desmethylvenlafaxine hydrochloride salt, an amorphous O-desmethylvenlafaxine sulfuric acid salt, an amorphous O-desmethylvenlafaxine citrate salt, an amorphous O-desmethylvenlafaxine maleate salt, an amorphous O-desmethylvenlafaxine mesylate salt, an amorphous O-desmethylvenlafaxine mandelate salt, an amorphous O-desmethylvenlafaxine malic acid salt, an amorphous O-desmethylvenlafaxine quinic acid salt, an amorphous O-desmethylvenlafaxine tartrate salt, and combinations thereof.
